(19) United States
(12) Plant Patent Application Publication
(10) Pub. No.: US 2004/0168238 P1
(43) Pub. Date: Aug. 26, 2004

(54) Artichoke plant named BCV 8-11

(76) Inventor: Medhat Chahbandar, Torreilles (FR)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/371,510

(22) Filed: Feb. 20, 2003

(51) Int. Cl. A01H 5/00

(52) U.S. Cl. PLT/258

(57) ABSTRACT

A new and distinct cultivar of Artichoke plant named ‘BCV 8-11’, having bracht exhibiting a much darker and vibrant violet hue, reduced pubescence, sterility, more tender fruit and greater production of fruit buds.

### BACKGROUND OF THE INVENTION

The present invention comprises a new and distinct cultivar of artichoke plant, botanically known as Cynara scolymus L., herein referred to by the cultivar name ‘BCV 8-11’. This variety is characterized by bracht having a much darker and vibrant violet or purple hue, reduced pubescence with a more tender fruit, it is sterile, and has a greatly enhanced prolificity, i. e., a greater production of fruit buds per plant, than the parent and other varieties known to the inventor.

Cynara scolymus L., commonly known as Globe artichoke, is a perennial herb and is a member of the family Asteraceae, also known as the Compositae family. Globe artichokes comprise: leaves, which are pinnately lobed, but primarily spineless; globose capitula composed of overlapping layers of large involucral bracts; and receptacles, which are enlarged and fleshy. Globe artichoke plants are essentially grown for the production of the immature flower heads (or buds), which are considered vegetable delicacies. Fresh artichokes may be steamed, boiled, or baked after which the fleshy receptacle, inner and outer bracts, and parts of the floral stem may be eaten.

The new cultivar is a product of a planned breeding program carried out by the inventor, M. Chanbandar near Perpignan, France. It was identified by visual selection of a variety derived from open pollination of the artichoke variety Violet de Provence. After several generations under conditions that caused somaclonal variability, it was further selected. Further asexual tissue culture propogation revealed that the new cultivar was stable. It was demonstrated that the combination of characteristics as herein disclosed for the new cultivar are firmly fixed and retained through successive generations of asexual reproduction.

### BRIEF DESCRIPTION OF THE INVENTION

The following traits have been repeatedly observed and are determined to be basic characteristics of ‘BCV 8-11’. These traits in combination distinguish the Artichoke as a new and distinct cultivar from the parent and any other known varieties. The bracts of ‘BCV 8-11’ have much darker and vibrant violet or purple hue than the parent or any other variety. It has reduced pubescence, is sterile, produces a more tender fruit, and has shown to exhibit a greatly enhanced prolificity, i. e., a greater production of fruit buds per plant.

‘BCV 8-11’ has not been observed under all possible environmental conditions. The phenotype of the new cultivar may vary with variations in environment such as temperature, humidity, light intensity and day length, without any change in the genotype. However, the following observations, measurements and values, except for seed observations, describe the new cultivar as grown in Lompoc, Calif. under conditions which closely approximate those generally used in horticultural practice. Seed observations were carried out in Perpignan, France.

Lompoc is located on California's central coast in Santa Barbara County. Conditions can vary in the summer months. Air temperatures can range between the low 60's to 80's. The relative humidity is generally high. Prevailing winds are northwesterly and rainfall averages 12 inches per year.

In the following description, holding quality was measured by the physical appearance of the head. This includes the head's appearance following 3 and 7 day storage periods in cold storage at 34° F. The head's exterior (oxidation) was observed at each of the two observation points. Browning and blackening of plant tissue was evaluated as light, moderate and extreme. Overall storage response was measured by observing heads following 3 and 7 day cold storage periods. These observations concentrated on visible color variability and/or presence of lesions or other cosmetic anomalies. Leaf ratio (L/W) was determined by dividing representative leaf sample lengths by their width. Finally, head response (weather) was determined by observing the heads at maturity. These field observations focus on the presence or absence of bronzing, necrotic or chlorotic lesions or any abiotic responses to environmental conditions. AFLP (DNA analysis) was conducted on each parental selection.

### BRIEF DESCRIPTION OF THE DRAWINGS

This new variety is illustrated by the accompanying photographic drawings, with such colors in the photograph being as true to those of the plant as can be reasonably obtained from conventional photographic procedures. In such photographs:

FIG. 1 shows a side elevational view of ‘BCV 8-11’s fruit in full color.

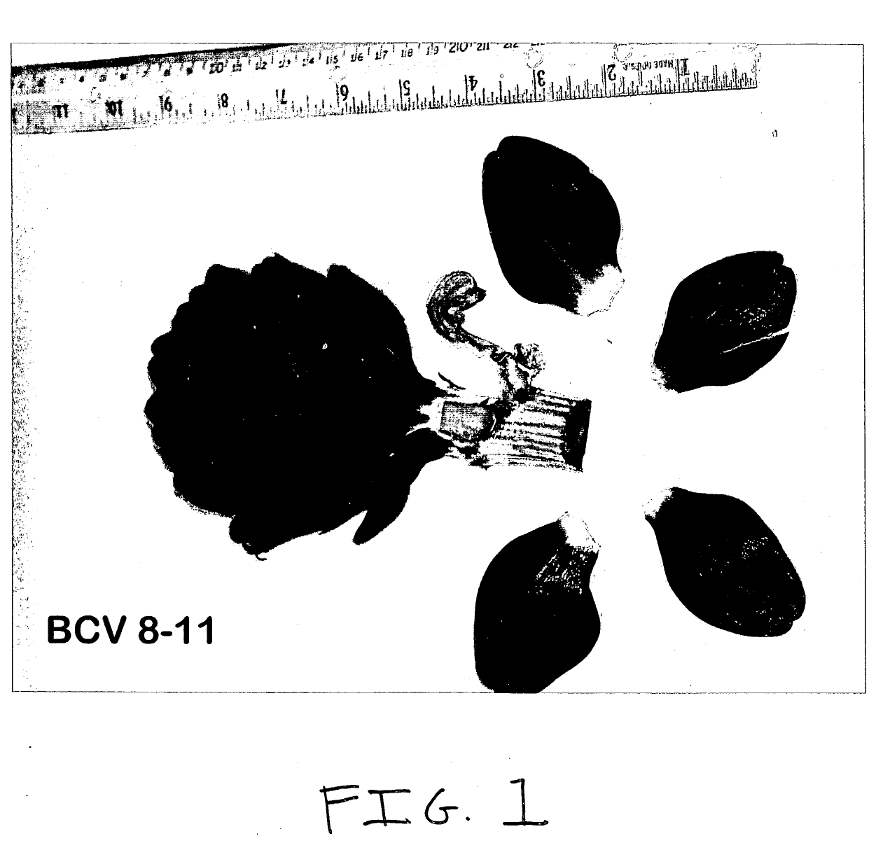

FIG. 2 shows a side elevational view of ‘BCV 8-11’s foliage in full color.

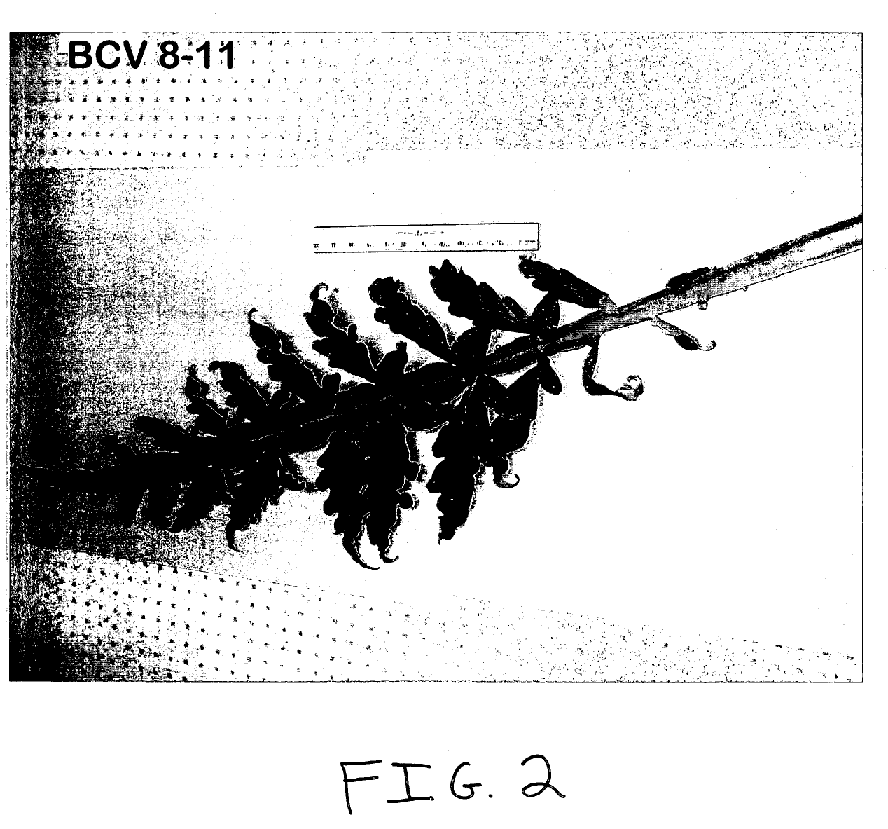

FIG. 3 shows a close-up view of the vertical cross-section of ‘BCV 8-11’s fruit in full color.

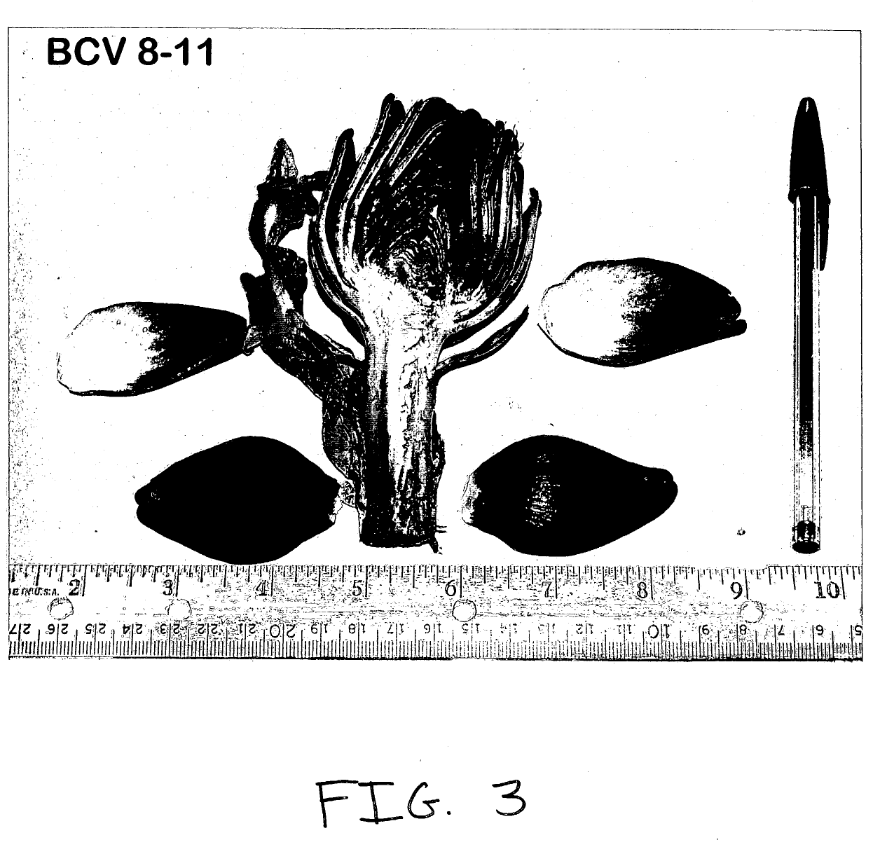

### DETAILED DESCRIPTION OF THE INVENTION

The following observations, measurements, and values describe the new artichoke plant is based upon observations of plants grown in Lompoc, Calif. The data was collected from tissue culture plants grown in Perpignan and acclimatized in California. Selections were made in outdoor field settings. The plants were grown in the soil in rows where row spacing (bed centers) was at 80 inches, and individual plant spacing at 24 inches. The plants were then planted in July and the observations described herein taken in December of the next year. Color references are measured against The Royal Horticultural Society Colour.

General:

Parentage.—Selections from variety Violet de Provence.

Classification.—Cynara Scolymus L. cv BCV 8-11.

Propagation.—Asexual production by tissue culture.

Height.—94.0-121.9 cm; average 108.7 cm.

Width.—182.9-241.3 cm; average 213.9 cm.

Form.—Full.

Growth habit.—Upright with some open.

Plant vigor.—Slight.

Side shoots:

Development.—Moderate.

Color.—Yellow green group 147 b.

Main stem diameter.—5.4-7.1 cm; average 6.6 cm.

Capitulum:

Size (Primary).—Approximately 9.0 cm.

Shape.—Oval with reflexive bracts.

Weight.—150-200 gm.

Texture.—Smooth, Felt-like.

Fragrance.—Mild with vegetative or grassy overtones.

Bract:

Length.—Approximately 7 cm.

Width.—Approximately 5 cm.

Shape.—Triangle to ovate bract, medium width, long depth of base, thick, outwardly reflexive bract.

Texture.—Smooth with more texture like felt.

Number.—102 per head.

Color (inner).—Grayed purple group 187 b-c.

Color (outer).—Grayed purple group 187a-b.

Firmness.—Less Firm and fleshy with thinner basal thickness.

Spinosity.—None, or minimal — less than 1 mm.

Basal thickness.—6-17 mm.

Miscellaneous:

Heart description.—Concave full, well developed with thick bract connection.

Receptacle thickness.—0.9 cm.

Heart color.—Yellow green group 150 b-c.

Florets.—Sterile.

Pappus length.—0.8 cm.

Pappus color.—Yellow green group 150 d.

Head firmness.—Firm, heads are dense and solid.

Gloss.—Less glossy than Green Globe.

Cold storage response.—Minor brown coloration on the cut stem. Overall less glossy.

Head response (weather).—After frosts the outer epidermal layer would crinkle and blister, but not discolored.

Foliage:

Shape.—Long strap-like leaves, very lobed and moderately dentate.

Density.—Dense.

Length.—Approximately 116.8-147.3 cm.

Width.—Approximately 34.3-47.0 cm.

Distance between main lobes.—Approximately 6.8-8.9 cm.

Leaf ratio.—Approximately 2.7-3.7 cm.

Leaf area.—Approximately 1,714-2,561 cm sq.

Upper leaf surface color.—Green group 139 b-c.

Lower leaf surface color.—Green group 138 b-c.

Texture.—Slightly but uniformly textured.

Pubescence.—Leaves more pubescent.

Lobe width.—Approximately 8.6-11.4 cm.

Lobe indentation.—Approximately 4.2-4.4 cm.

Petiole length.—Approximately 20.3-25.4 cm.

Petiole width.—Approximately 2.5-3.8 cm.

Petiole thickness.—Approximately 1.0-2.1 cm.

Seeds:

Shape.—Ovoid asymmetrical.

Number.—Very irregular.

Length.—Approx 6 mm.

Color.—White yellow.

What is claimed is:

1. A new and distinct variety of artichoke plant substantially as shown and described, characterized particularly as to novelty by the characteristics listed above.
